(19) United States
(12) Patent Application Publication
(10) Pub. No.: US 2024/0108867 A1
(43) Pub. Date: Apr. 4, 2024

(54) BALLOON CATHETER

(71) Applicant: GOODMAN CO., LTD., Aichi (JP)

(72) Inventor: Yuta Nakamura, Aichi (JP)

(73) Assignee: GOODMAN CO., LTD., Aichi (JP)

(21) Appl. No.: 18/538,115

(22) Filed: Dec. 13, 2023

Related U.S. Application Data
(63) Continuation of application No. PCT/JP22/20770, filed on May 19, 2022.

(30) Foreign Application Priority Data
Jun. 17, 2021 (JP) 2021-101067

(51) Int. Cl. A61M 25/10; A61M 25/00

(52) U.S. Cl. CPC A61M 25/104; A61M 25/0043

(57) ABSTRACT

A balloon catheter includes a balloon that is inflatable and deflatable, includes a straight tube portion and a linear protrusion that protrudes from an outer surface of the straight tube portion and extends in an axial direction of the straight tube portion. The linear protrusion includes a slope portion wherein a ridge slopes down to the outer surface, the slope portion includes a first slope portion in which the ridge slopes down to the outer surface toward a tip-end side, and the linear protrusion has a projection height from the outer surface that becomes lower toward the tip-end side in the axial direction within a region between a base-end side edge of the first slope portion and a tip-end side edge of the linear protrusion.

### CROSS-REFERENCE TO RELATED APPLICATION

The present application is a continuation of PCT International Application No. PCT/JP2022/020770 filed on May 19, 2022 which claims the benefit of priority from Japanese Patent Application No. 2021-101067 filed on Jun. 17, 2021, the entire contents of which are incorporated herein by reference.

### BACKGROUND

### Technical Field

The present disclosure relates to a balloon catheter.

### Description of Related Art

A balloon catheter has been used in treatment such as PTA (percutaneous transluminal angioplasty) and PTCA (percutaneous transluminal coronary angioplasty). The balloon catheter includes an inflatable and deflatable balloon on a tip-end side of the balloon catheter. The balloon in a deflated state may be inserted into a site of lesion at which a vessel is narrowed or blocked due to a lesion (hereinafter the site of lesion may be referred to as a lesion site) and then inflated to widen the section of the vessel.

The balloon catheter may include linear portions that extend in an axial direction of the balloon catheter on an outer surface of the balloon. The linear portions may protrude from the outer surface of the balloon. When the balloon is inflated at the lesion site, the linear portions may dig into the lesion and incisions may be made in the lesion. The lesion may easily break from the incisions and thus the lesion site may easily widen.

An example of such a balloon catheter is disclosed in Patent Literature 1. Linear portions in the example protrude from an outer surface of a straight tube portion of a balloon. The straight tube portion has a cylindrical shape and a maximum diameter when the balloon is inflated. The linear portions extend for an entire length of the straight tube portion in the axial direction. The height of each linear portion above the outer surface of the straight tube is constant for an entire length of the linear portion.
Patent Literature 1: Japanese Unexamined Patent Application Publication No. 2009-112361.

Because the height of each linear portion on the outer surface of the straight tube portion is constant, an outer diameter of the straight tube portion may be constantly large for the entire length of the straight tube portion. Therefore, insertability of the balloon during insertion of the balloon into the lesion site may decrease.

In the configuration in which the height of each linear portion is constant, a large area of the linear portion in a longitudinal direction may contact the lesion at a time. In such a situation, the linear portions may not easily dig into the lesion and thus proper incisions may not be made. Namely, the lesion may not properly break from the incisions and thus the lesion site may not properly widen.

### SUMMARY

One or more embodiments of the present discloser provide a balloon catheter that properly widen a lesion site without reducing insertability of a balloon.

A balloon catheter according to a first aspect includes a balloon (or inflatable balloon) disposed on a tip-end side of the balloon catheter. The balloon is inflatable and deflatable. The balloon includes a straight tube portion having a cylindrical shape, and a diameter of the inflatable balloon is largest at the straight tube portion among portions of the inflatable balloon in a state where the balloon is inflated. The balloon catheter includes a linear protrusion that protrudes from an outer surface of the straight tube portion and extends in an axial direction of the straight tube portion. The linear protrusion includes a ridge at a projected end of the linear protrusion. The ridge extends in a longitudinal direction of the linear protrusion. The linear protrusion includes a slope portion that extends over at least a part of the linear protrusion along the longitudinal direction, wherein the ridge slopes down to the outer surface in the slope portion. The slope portion includes a first slope portion in which the ridge slopes down to the outer surface toward the tip-end side. The linear protrusion has a projection height from the outer surface that becomes lower toward the tip-end side in the axial direction within a region between a base-end side edge of the first slope portion and a tip-end side edge of the linear protrusion.

In the first aspect, the linear protrusion that extends in the axial direction is on the straight tube portion of the balloon. The linear protrusion includes the slope portion. In the slope portion, the ridge slopes down to the outer surface of the straight tube portion. When the slope portion is placed in a lesion site and the balloon is inflated, the ridge in the slope portion locally contacts a lesion at an angle. According to the configuration, the linear protrusion can easily dig into the lesion. Therefore, an incision can be easily made in the lesion and thus the lesion properly breaks from the incision.

The slope portion includes the first slope portion in which the ridge slopes down to the outer surface toward the tip-end side. Because the slope portion includes the first slope portion, the height above the outer surface of the straight tube portion is smaller on the tip-end side than a base-end side in the portion of the linear protrusion between a tip-end side edge of the linear protrusion and the base-end side edge of the first slope portion. According to the configuration, insertability of the balloon into the lesion site is less likely to decrease.

In a balloon catheter according to a second aspect, a tip-end side edge of the first slope portion matches the tip-end side edge of the linear protrusion. The ridge may join the outer surface at the tip-end side edge of the first slope portion.

According to the second aspect, a step is less likely to form at the tip-end side edge of the linear protrusion and thus the balloon is less likely to be caught by the lesion due to the step during insertion of the balloon into the lesion site. Therefore, the balloon can be easily inserted into the lesion site.

In a balloon catheter according to a third aspect, the linear protrusion in the first or the second aspect may include a non-slope portion that joins the base-end side edge of the first slope portion. The ridge in the non-slope portion may extend parallel to the outer surface.

In the third aspect, the linear protrusion may include the non-slope portion that joins the base-end side edge of the first slope portion. In the non-slope portion, the ridge may extend parallel to the outer surface of the straight tube portion. According to the configuration, the lesion site may be widened by the first slope portion, the balloon may be advanced to the tip-end side, and then the lesion site may be further widened by the non-slope portion. Therefore, the lesion site may be uniformly widened at the end of the procedure.

In a balloon catheter according to a fourth aspect, the tip-end side edge of the linear protrusion is closer to a base-end side than is the tip-end side edge of the straight tube portion, and the straight tube portion in any one of the first to third aspect may include a no-protrusion region that does not include the linear protrusion. The no-protrusion region is closer to the tip-end side than is the linear protrusion.

In the fourth aspect, the no-protrusion region of the straight tube portion in which the linear protrusion may not exist is closer to the tip-end side than is the linear protrusion. Therefore, the insertability of the balloon into the lesion site may improve. The no-protrusion region may be more flexible because no protrusion exists in this section. This may also improve the insertability. If the lesion site is narrowed, the no-protrusion may be first inserted into the lesion site, the balloon may be inflated to slightly widen the lesion site, the balloon may be advanced to the tip-end side, and then the lesion site may be widened by the first slope portion.

In a balloon catheter according to a fifth aspect, the first slope portion in any one of the first to the fourth aspect may include a tip-end side slope region and a base-end side slope region that is disposed closer to the base-end side than is the tip-end side slope region. A slope angle of the ridge to the outer surface in the tip-end side slope region may be different from a slope angle of the ridge to the outer surface in the base-end side slope region.

In the fifth aspect, the first slope portion may include the tip-end side slope region and the base-end side slope region in which the slope angles of the ridge are different from each other. According to the configuration, a variety of ways of widening the lesion site can be provided. For example, the tip-end side slope region may be first used and then the base-end side slope region may be used to widen the lesion site by the first slope portion.

In a balloon catheter according to a sixth aspect, the slope angle of the ridge in the tip-end side slope region in the fifth aspect may be smaller than the slope angle of the ridge in the base-end side slope region.

Because the slope angle of the ridge in the tip-end side slope region is smaller in the sixth aspect, an overall height of the linear protrusion can be reduced. Therefore, the insertability of the balloon may be less likely to decrease. Because the slope angle of the ridge in the base-end side slope region may be set larger, the linear protrusion can further easily dig into the lesion. Therefore, the lesion site can be properly widened.

In a balloon catheter according to a seventh aspect, the linear protrusion in the first or the second aspect may include a second slope portion that extends from the base-end side edge of the first slope portion toward the base-end side. The ridge in the second slope portion slopes down in a direction same as a direction in which the first slope portion slopes down. The ridge at a tip-end side edge of the second slope portion is closer to the outer surface than is the ridge at the base-end side edge of the first slope portion.

In the seventh aspect, the linear protrusion may include the second slope portion on the base-end side of the first slope portion. The ridge slopes down in the same direction in the first slope portion and the second slope portion. The ridge at the tip-end side edge of the second slope portion is closer to the outer surface of the straight tube portion than is the ridge at the base-end side edge of the first slope portion. According to the configuration, a corner may be defined at the base-end side edge of the first slope portion. With the corner, the linear protrusion can further easily dig into the lesion.

In a balloon catheter according to an eighth aspect, the linear protrusion in the first or the second aspect may include a third slope portion that is closer to the base-end side than is the first slope portion. The ridge in the third slope portion may slope down to the outer surface toward the base-end side.

In the eighth aspect, the ridge of the linear protrusion in the third slope portion that is closer to the base-end side than is the first slope portion slopes down to the outer surface of the straight tube portion toward the base-end side. According to the configuration, the balloon with the linear protrusion is less likely to be caught by a vessel wall or the lesion in the body during removal of the balloon from the body. Therefore, the balloon can be easily removed from the body.

In a balloon catheter according to a ninth aspect, the third slope portion in the eighth aspect may extend from the base-end side edge of the first slope portion toward the base-end side. The ridge in the third slope portion may join the ridge in the first slope portion at a border between the first slope portion and the third slope portion.

Because the ridge in the third slope portion in the ninth aspect may join the ridge in the first slope portion at the border between the first slope portion and the third slope portion, a corner is defined by the ridge in the first slope portion and the ridge in the third slope portion at the border between the first slope portion and the third slope portion. With the corner, the linear protrusion can further easily dig into the lesion.

### BRIEF DESCRIPTION OF DRAWINGS

FIG. 1 is a schematic overall side view illustrating a configuration of a balloon catheter.

FIG. 2 is a cross-sectional view of a balloon and an outer tube cut along a long axis of the balloon catheter illustrating the balloon that is in an inflated state and portions of the balloon catheter adjacent to the balloon.

FIG. 3A is a side view of the balloon catheter illustrating a configuration of the balloon in the inflated state and portions of the balloon catheter adjacent to the balloon.

FIG. 3B is a cross-sectional view cut along line A-A in FIG. 3A.

FIG. 4A is a side view of the balloon catheter illustrating a configuration of the balloon in a deflated state and portions of the balloon catheter adjacent to the balloon.

FIG. 4B is a cross-sectional view cut along line B-B in FIG. 4A.

FIGS. 5A to 5E are explanatory views illustrating a method of using the balloon catheter.

FIGS. 6A to 6D are explanatory views illustrating modifications of protrusions.

FIGS. 7A to 7C are explanatory views illustrating modifications of the protrusions.

FIGS. 8A and 8B are explanatory views illustrating modifications of the protrusions.

FIGS. 9A to 9C are explanatory views illustrating modifications of the protrusions.

### DETAILED DESCRIPTION

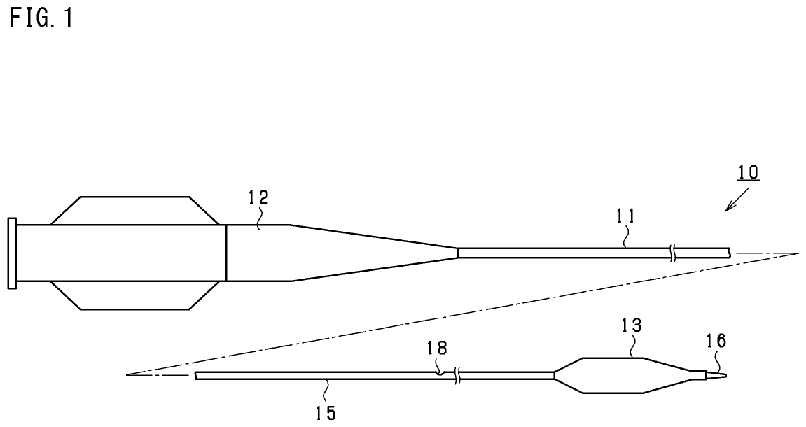

A balloon catheter 10 will be described with reference to the drawings. First, an overall configuration of the balloon catheter 10 will be described with reference to FIG. 1.

As illustrated in FIG. 1, the balloon catheter 10 includes a catheter tube 11, a hub 12, and a balloon 13. The hub 12 is coupled to a proximal end (a base end) of the catheter tube 11. The balloon 13 is coupled to a distal-end side (a tip-end side) of the catheter tube 11.

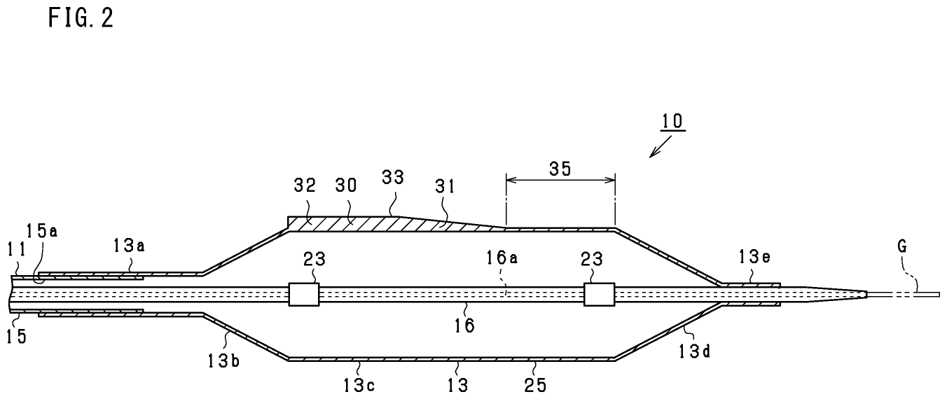

The catheter tube 11 includes an outer tube 15 and an inner tube 16 that is inserted in the outer tube 15. The outer tube 15 is made of a resin material and formed in a tubular shape. The outer tube 15 includes a fluid lumen 15a inside (see FIG. 2). The fluid lumen 15a extends throughout the outer tube 15 in an axial direction of the outer tube 15. The outer tube 15 incudes a distal end 15b coupled to the balloon 13 and a proximal end coupled to the hub 12. The fluid lumen 15a is communicated with an internal space of the hub 12 and an internal space of the balloon 13. For inflation and deflation of the balloon 13, a compressed fluid is passed through the fluid lumen 15a of the outer tube 15.

The outer tube 15 may be composed of multiple tubes that are in line in the axial direction and coupled to one another. One of the tubes on the proximal side may be made of a metal material and another one of the tubes on the distal side may be made of a resin material.

The inner tube 16 is made of a resin material and formed in a tubular shape. The inner tube 16 includes a guide wire lumen 16a inside (see FIG. 2). The guide wire lumen 16a extends through the inner tube 16 in an axial direction of the inner tube 16. The inner tube 16 includes a proximal end coupled to a portion of the outer tube 15 in the middle with respect to the axial direction. A distal-end side of the inner tube 16 projects toward the distal side than the distal end of the outer tube 15. The distal-end side of the inner tube 16 is passed through the internal space of the balloon 13.

A guide wire G is passed through the guide wire lumen 16a. The inner tube 16 includes a proximal-end opening 18 that is located in the middle of the balloon catheter 10 with respect to an axial direction of the balloon catheter 10. That is, the balloon catheter 10 is classified as an RX-type catheter. The proximal-end opening 18 may be located in the proximal end of the balloon catheter 10. The balloon catheter 10 having such a configuration may be classified as an over-the-wire-type catheter.

Next, the balloon 13 and portions of the balloon catheter 10 adjacent to the balloon 13 will be described with reference to FIGS. 2 to 4B.

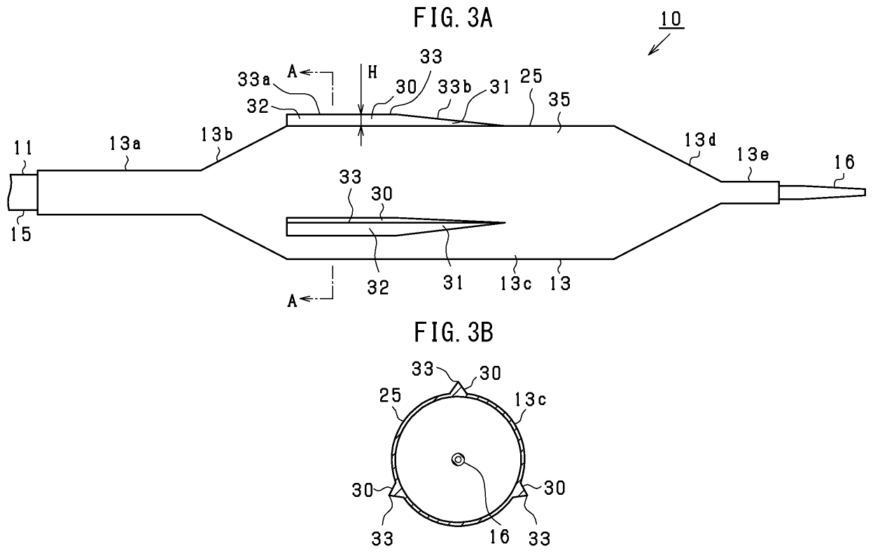

The balloon 13 is made of a thermoplastic resin material such as polyamide elastomer. As illustrated in FIGS. 2 to 3B, the balloon 13 has a cylindrical shape (a tubular shape) with a cross section that is circular as a whole. The balloon 13 includes a proximal-side joint portion 13a, a proximal-side tapered portion 13b, a straight tube portion 13c, a distal-side tapered portion 13d, and a distal-side joint portion 13e, which are arranged in this sequence from the proximal side to the distal side.

The proximal-side joint portion 13a is joined to the distal end of the outer tube 15. The proximal-side tapered portion 13b has a tapered shape with a diameter that increases from the distal end of the proximal-side joint portion 13a toward the distal side. The straight tube portion 13c has a round tubular shape that extends from a distal end of the proximal-side tapered portion 13b toward the distal side with a constant diameter. The straight tube portion 13c is a portion, the diameter of which in the inflated state is the largest. The distal-side tapered portion 13d has a tapered shape with a diameter that decreases from the distal end of the straight tube portion 13 toward the distal side. The distal-side joint portion 13e is joined to the distal-end side of the inner tube 16. The proximal-side tapered portion 13b, the straight tube portion 13c, and the distal-side tapered portion 13d consist of an inflatable and deflatable portion that inflates and deflates.

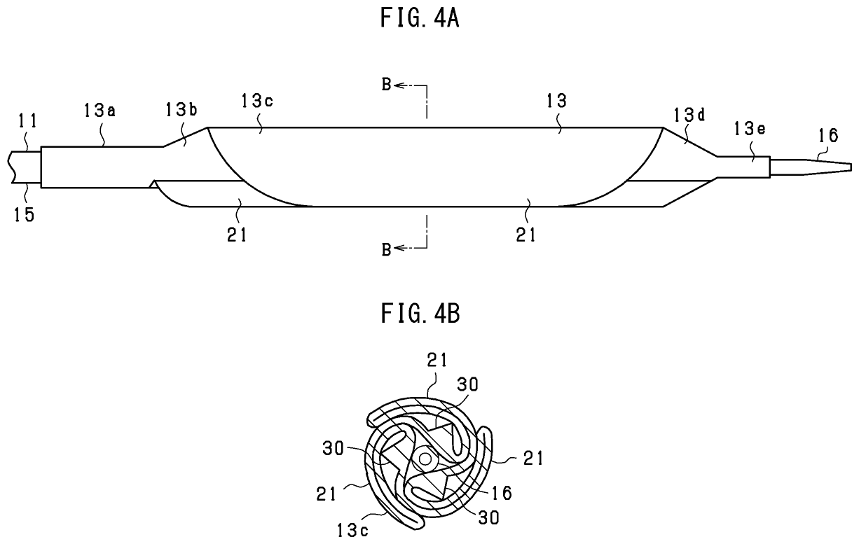

When the compressed fluid is supplied to the internal space of the balloon 13 via the fluid lumen 15a of the outer tube 15, the balloon 13 inflates. When a negative pressure is applied to the fluid lumen 15a of the outer tube 15 and the compressed fluid flows out of the internal space of the balloon 13, the balloon 13 deflates. As illustrated in FIGS. 4A and 4B, multiple wings 21 (three wings 21 in one or more embodiments) appear when the balloon 13 deflates. The wings 21 are at predefined intervals (specifically, equal intervals) in a circumferential direction of the balloon 13. The wings 21 appear in the inflatable and deflatable portion of the balloon 13 to extend in the axial direction. When the balloon 13 is in the deflated state, the wings 21 are folded along the circumferential direction and wrapped around the inner tube 16.

Two contrast rings 23 are attached to the inner tube 16 inside the balloon 13. The contrast rings 23 are for improving visibility of the balloon 13 in x-ray images to easily position the balloon 13 to a target spot for treatment.

The balloon catheter 10 includes linear protrusions 30 that protrude from an outer surface 25 of the straight tube portion 13c. The protrusions 30 are for making incisions in a lesion when the balloon 13 is inflated to break the lesion. Even if the lesion is hardened due to calcification or any other causes, the lesion breaks from the incisions made by the protrusions 30 of the balloon catheter 10. The balloon catheter 10 is configured as a balloon catheter having a scoring function.

The protrusions 30 will be described in detail. As illustrated in FIGS. 2 to 3B, the protrusions 30 are on the straight tube portion 13 of the balloon 13. Specifically, the protrusions 30 are exclusively on the straight tube portion 13c. The protrusion 30 protrudes from the outer surface 25 of the straight tube portion 13c. The protrusions 30 linearly extend in the axial direction of the straight tube portion 13c (hereinafter may be simply referred to as the axial direction). The protrusions 30 are at predefined intervals (specifically, at equal intervals) in the circumferential direction of the straight tube portion 13c. In one or more embodiments, three protrusions 30 are at 120° intervals. The protrusions 30 and the balloon 13 are integrally molded.

Distal edges of the protrusions 30 are at the same position with respect to the axial direction. The distal edges of the protrusions 30 are closer to the proximal side than a distal edge of the straight tube portion 13c. Namely, the protrusions 30 do not exist in a section of the straight tube portion 13c closer to the distal side than the distal edges of the protrusions 30. The section is defined as a no-protrusion region 35. In one or more embodiments, a length of the no-protrusion region 35 measuring in the axial direction is ⅓ of a length of the straight tube portion 13c measuring in the axial direction. The length of the no-protrusion region 35 may not be limited to ⅓ of the length of the straight tube portion 13 and may be greater or less than ⅓ of the length of the straight tube portion 13c. Proximal edges of the protrusions 30 are at the same position as the proximal edge of the straight tube portion 13c with respect to the axial direction.

Each protrusion 30 has a triangular cross section (cut along a plane perpendicular to the longitudinal direction of the protrusion 30) that protrudes outward. Specifically, the cross section of each protrusion 30 is triangular for the entire length of the protrusion 30. In one or more embodiments, the cross section of each protrusion 30 is in a triangular shape that protrudes outward. The cross section of the protrusion 30 is not limited to the triangular shape. The cross section of the protrusion 30 may be in a semicircular shape or in any other shape. The protrusion 30 includes a ridge 33 at a projected end of the protrusion 30. The ridge 33 extends for the entire length of the protrusion 30.

The protrusion 30 includes a slope portion 31 on the distal side and a non-slope portion 32 that joins the slope portion 31 on the proximal side. The slope portion 31 includes the distal edge of the protrusion 30. The non-slope portion 32 includes the proximal edge of the protrusion 30. The slope portion 31 may correspond to a first slope portion.

The ridge 33 of the protrusion 30 is angled to the outer surface 25 of the straight tube portion 13c (i.e., to the axial direction) in the slope portion 31. The ridge 33 in the slope portion 31 slopes down to the outer surface 25 of the straight tube portion 13c toward the distal side. In the slope portion 31 (or in a portion of the protrusion 30 between the distal edge of the protrusion 30 and a proximal edge of the slope portion 31), a height H of the protrusion 30 above the outer surface 25 of the straight tube portion 13c gradually decreases toward the distal side. The ridge 33 joins the outer surface 25 of the straight tube portion 13c at the distal edge of the slope portion 31. In the slope portion 31, a slope angle of the ridge 33 to the outer surface 25 of the straight tube portion 13c is constant for an entire length of the slope portion 31.

In the non-slope portion 32, the ridge 33 of the protrusion 30 extends parallel to the outer surface 25 of the straight tube portion 13c. In the non-slope portion 32, the height H of the protrusion 30 above the outer surface 25 of the straight tube portion 13c is constant for the entire length of the non-slope portion 32. A section of the ridge 33 in the non-slope portion 32 (hereinafter may be referred to as a first section 33a of the ridge 33) extends from the proximal edge of a section of the ridge 33 in the slope portion 31 (hereinafter may be referred to as a second section 33b of the ridge 33) toward the proximal side. The second section 33b of the ridge 33 joins the first section 33a of the ridge 33 at a border between the slope portion 31 and the non-slope portion 32. Therefore, the height H of the protrusion 30 above the outer surface 25 of the straight tube portion 13c in the non-slope portion 32 is equal to the height H of the protrusion 30 above the outer surface 25 of the straight tube portion 13c at the proximal edge of the slope portion 31.

The length of the slope portion 31 measuring in the axial direction is about equal to, but not limited to, the length of the non-slope portion 32 measuring in the axial direction. The length of the slope portion 31 measuring in the axial direction may be greater than or less than the length of the non-slope portion 32 measuring in the axial direction. The length of the slope portion 31 and the length of the non-slope portion 32 measuring in the axial direction are about equal to, but not limited to, a length of the non-protrusion region 35 measuring in the axial direction. The length of the slope portion 31 and the non-slope portion 32 measuring in the axial direction may be greater than or less than the lengths of the non-protrusion region 35 measuring in the axial direction.

As described earlier, when the balloon 13 is deflated, multiple wings 21 appear in the inflatable and deflatable portion (the straight tube portion 13c and the tapered portions 13b and 13d). The wings 21 are folded in the circumferential direction of the balloon 13. As illustrated in FIGS. 4A and 4B, the protrusions 30 on the straight tube portion 13c correspond to the wings 21, respectively. The protrusions 30 are in inner spaces defined by the folded wings 21, respectively. Namely, each protrusion 30 is covered with the corresponding wing 21 from an outer side when the balloon 13 is in the deflated state.

A method of producing the balloon 13 will be briefly described.

First, a parison having a tubular shape is prepared by extrusion for a base of the balloon 13. Triangular elongated protrusions are formed on a lateral surface of the parison such that the elongated protrusions extend in the axial direction of the parison. Each elongated protrusion has a triangular cross section. Multiple elongated protrusions (three elongated protrusions in one or more embodiments) are at equal intervals in the circumferential direction of the parison.

Next, the parison is stretched in the longitudinal direction and formed into a shape of the balloon 13 under predefined conditions by blow molding using a die. The die has a cavity corresponding to the shape of the balloon 13 and grooves for holding the elongated protrusions. The parison is set in the cavity and the elongated protrusions are set in the grooves. Then, the blow molding is performed. During the blow molding, the parison is heated and stretched in the cavity of the die. Through the blow molding, the prison is stretched in two axial directions and the elongated protrusions are formed into the protrusions 30. Ends of the stretched parison are cut and the production of the balloon 13 is complete.

The method of producing the balloon 13 is not limited to the method described above. Any method may be used.

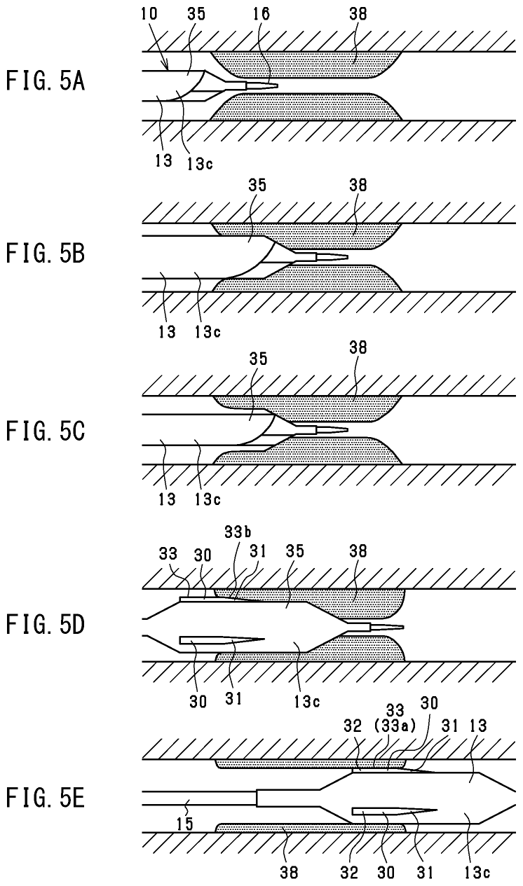

A method of using the balloon catheter 10 will be described. A procedure for widening a section of a blood vessel at a lesion site using the balloon catheter 10 will be described with reference to FIGS. 5A to 5E.

A guiding catheter is inserted into a sheath introducer, which is inserted in the blood vessel, until a front-end opening of the guiding catheter reaches a coronary ostium. The guide wire G is inserted into the guiding catheter and advanced toward a distal portion of the blood vessel via the coronary ostium and the lesion site.

Then, the balloon catheter 10 is inserted into the guiding catheter along the guide wire G. After the insertion, the balloon 13 in the deflated state is moved back and forth until the balloon 13 reaches the lesion site.

As illustrated in FIG. 5A, a lesion 38 in the blood vessel is relatively large and thus the lesion site is significantly narrowed. Further, the lesion 38 is calcified and hardened. In this case, the balloon 13 (the straight tube portion 13c) with the protrusions 30 may not be properly advanced to the lesion site.

As illustrated in FIG. 5A, the no-protrusion region 35 of the straight tube portion 13c closer to the distal side than the protrusions 30 is inserted into the lesion site. The no-protrusion region 35 has an outer diameter less than the outer diameter of other sections of the straight tube portion 13c with the protrusions 30. Further, the no-protrusion region 35 has flexibility greater than flexibility of the other sections with the protrusions 30. Namely, the no-protrusion region 35 has insertability greater than insertability of the other sections with the protrusions 30. Therefore, the no-protrusion region 35 can be easily inserted into the lesion site even if the lesion 38 is large.

The compressed fluid is supplied to the balloon 13 with the no-protrusion region 35 placed in the lesion site. As illustrated in FIG. 5B, the balloon 13 slightly inflates from the deflated state and thus the lesion site slightly widens. This allows the section of the straight tube portion 13c with the slope portions 31 to enter into the lesion site.

As illustrated in FIG. 5C, the balloon 13 is deflated. The balloon 13 in the deflated state is advanced to the distal side until the section of the straight tube portion 13c with the slope portions 31 is placed in the lesion site. Because the height H of the slope portions 31 above the outer surface 25 of the straight tube portion 13c decreases toward the distal side, the insertability of the straight tube portion 13c into the lesion site is less likely to decrease although the protrusions 30 protrude from the straight tube portion 13c.

As illustrated in FIG. 5D, the balloon 13 is inflated. The slope portions 31 of the protrusions 30 are pressed against the lesion 38 and incisions (cuts) are made in the lesion 38 by the slope portions 31. The lesion 38 breaks from the incisions and thus the lesion site widens. Because the second sections 33b of the ridges 33 slope, the second sections 33b of the ridges 33 locally contact the lesion 38 at an angle. According to the configuration, the protrusions 30 (the slope portions 31) can easily dig into the lesion 38. Therefore, incisions can be easily made in the lesion 38 even if the lesion 38 is hardened and thus the lesion 38 properly breaks from the incisions and the lesion site widens.

The balloon 13 is further advanced to the distal side until the non-slope portions 32 of the protrusions 30 are placed in the lesion site, more specifically, where the incisions are made in the lesion 38 by the slope portions 31 and the section of the blood vessel is widened. Then, the balloon 13 is inflated as illustrated in FIG. 5E. The non-slope portions 32 of the protrusions 30 pressed against the lesion 38 make incisions in the lesion 38. The lesion 38 further breaks from the incisions and thus the lesion site further widens. Because the second sections 33b of the ridges 33 extend parallel to the outer surface 25 of the straight tube portion 13c, the lesion site uniformly widens.

The inflation and deflation of the balloon 13 is repeated during advancement of the balloon 13 to the distal side as described above to widen the lesion site. After the lesion site is widened, the balloon 13 is deflated and removal of the balloon catheter 10 from the body is performed. This completes a series of steps.

As described above, the balloon catheter 10 may be used for treatment of a blood vessel in which the balloon catheter 10 is passed through the blood vessel. Examples of the blood vessel may include a coronary artery, a femoral artery, and a pulmonary artery. The balloon catheter 10 may be used in treatment of any vessels in living organisms other than blood vessels such as ureters and gastrointestinal tract or body cavities.

According to the configuration of the embodiments described above, the following advantageous effects can be achieved.

The ridges 33 of the protrusions 30 join the outer surface 25 of the straight tube portion 13c at the distal edges of the slope portions 31. According to the configuration, steps are less likely to be formed at the distal edges of the slope portions 31, that is, at the distal edges of the protrusions 30. Therefore, the balloon 13 is less likely to be caught by the lesion 38, which may occur due to such steps, during insertion of the balloon 13 to the lesion site. Namely, the balloon 13 is easily inserted into the lesion site. Further, the balloon 13 is less likely to be caught by a stent, if any, in the body.

Because protrusions 30 do not exist in the no-protrusion region 35 that is closer to the distal side than the protrusions 30 on the straight tube portion 13c, the insertability of the balloon 13 into the lesion site improves. Further, the distal-end side of the balloon 13 is more flexible. According to the configuration, during insertion of the balloon 13 into a curved section of a blood vessel, the balloon 13 can be more easily advanced along the curve.

In a configuration in which an entire area of each protrusion 30 is configured as the slope portion 31, if the slope angle of the ridge 33 to the outer surface 25 of the straight tube portion 13c is set larger to reduce cut resistant of the lesion 38 against the protrusion 30, the height of the protrusion 30 above the outer surface 25 at the proximal edge (e.g., the maximum height) may be significantly large. According to the configuration, the insertability of the balloon 13 into the lesion site may significantly decrease. If the slope angle of the ridge 33 is set smaller to reduce the height of the protrusion 30 above the outer surface 25, the protrusion 30 may not easily dig into the lesion 38.

According to the embodiments described earlier, each protrusion 30 includes not only the slope portion 31 but also the non-slope portion 32 that joins the proximal side of the slope portion 31. Therefore, the height of each protrusion 30 above the outer surface 25 can be maintained smaller while the slope angle of the ridge 33 is set relatively large. According to the configuration, the lesion site further properly widens although the insertability of the balloon 13 is further less likely to decrease.

The lengths of the slope portion 31 and the non-slope portion 32 of each protrusion 30 are about equal to each other. This configuration is preferable for further widening the lesion site by making incisions in the lesion 38 by the non-slope portions 32 after making incisions in the lesion 38 by the slope portions 31.

The lengths of the slope portion 31 and the non-slope portion 32 of each protrusion 30 are about equal to the length of the no-protrusion region 35 measuring in the axial direction. This configuration is preferable for slightly widening the lesion site by inserting the no-protrusion region 35 in the lesion site and then making incisions in the lesion 38 by the slope portions 31 and the non-slope portions 32.

The protrusions 30 and the balloon 13 (the straight tube portion 13c) are integrally molded. According to the configuration, displacement of the protrusions 30 is less likely to occur during making of incisions in the lesion 38 by the protrusions 30 on the balloon 13 that is inflated. Therefore, the incisions are properly made in the lesion 38.

The protrusions 30 are formed exclusively on the straight tube portion 13c of the balloon 13. According to the configuration, the insertability of the balloon 13 into the lesion site improves.

The disclosure is not limited to the above embodiments and the following embodiments may be included in the technical scope of the disclosure.

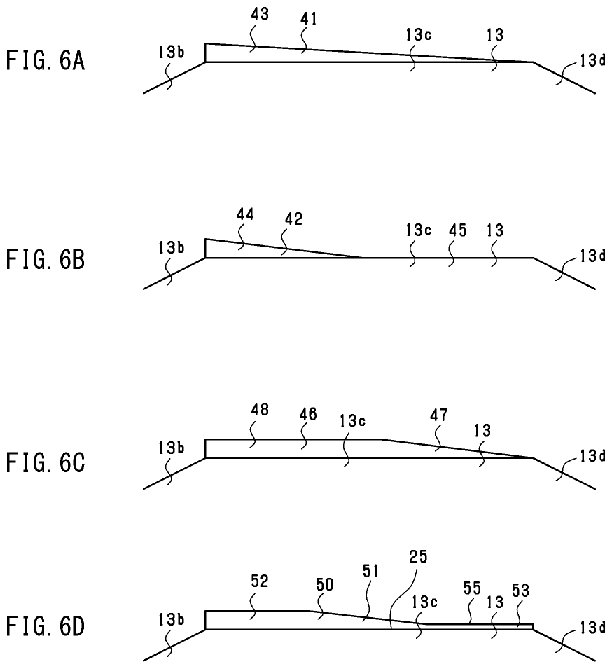

(1) The protrusions 30 are not limited to the above embodiments. Modifications of the protrusions 30 will be described with reference to FIGS. 6A, 6B, 6C and 6D, respectively.

Protrusions 41 and 42 illustrated in FIGS. 6A and 6B include slope portions 43 and 44 (each corresponding to a first slope portion), respectively. The slope portions 43 and 44 extend throughout the respective protrusions 41 and 42. The protrusions 41 illustrated in FIG. 6A extend for the entire length of the straight tube portion 13c in the axial direction. According to the configuration, pressure resistance of the straight tube portion 13c increases. The protrusions 42 illustrated in FIG. 6B include distal edges located closer to the proximal side than the middle of the straight tube portion 13c with respect to the axial direction. A section of the straight tube portion 13c closer to the distal side than the protrusions 42 is defined as a non-protrusion region 45. The length of the non-protrusion region 45 measuring in the axial direction is greater than the length of the no-protrusion region 35. Therefore, the insertability of the balloon 13 into the lesion site further improves.

Protrusions 46 illustrated in FIG. 6C include slope portions 47 (each corresponding to a first slope portion) and non-slope portions 48. The protrusions 46 extend for the entire length of the straight tube portion 13c in the axial direction, which is different from the above embodiments. According to the configuration, pressure resistance of the straight tube portion 13c increases, similarly to the straight tube portion 13c illustrated in FIG. 6A.

Protrusions 50 illustrated in FIG. 6D includes slope portions 51 (each corresponding to a first slope portion) and proximal-side non-slope portions 52 that are close to the proximal side than the slope portions 51. The protrusions 50 in this example further include distal-side non-slope portions 53 on the distal side of the slope portions 51. The distal edges of the distal-side non-slope portions 53 are aligned with the distal edge of the straight tube portion 13c with respect to the axial direction. Because the protrusions 50 extend for the entire length of the straight tube portion 13c in the axial direction, pressure resistance of the straight tube portion 13c increases, similarly to the straight tube portion 13c illustrated in FIG. 6A.

The distal-side non-slope portions 53 of the protrusions 50 have ridges 55 that extend parallel to the outer surface 25 of the straight tube portion 13c. The height of the distal-side non-slope portions 53 above the outer surface 25 of the straight tube portion 13c is equal to the height of the slope portions 51 at the distal edges of the slope portions 51. The height of each distal-side non-slope portion 53 is constant for the entire length of the distal-side non-slope portion 53. The height of each protrusion 50 is smaller on the distal side than on the proximal side in a portion of each protrusion 50 between the distal edge of the protrusion 50 and the proximal edge of the slope portion 51. Therefore, the insertability of the balloon 13 is less likely to decrease.

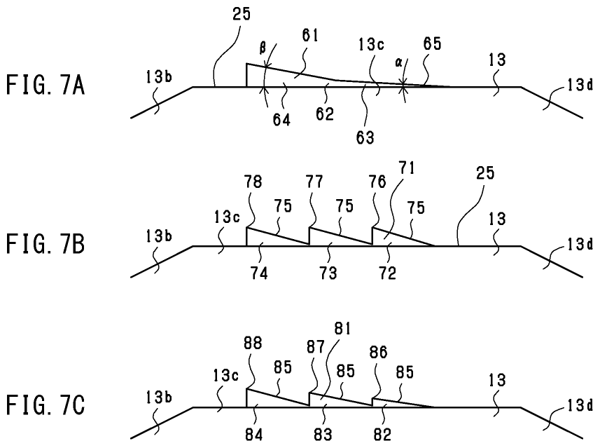

(2) The slope portion 31 of each protrusion 30 (corresponding to a first slope portion) in the above embodiments has the ridge 33, the angle of which to the outer surface 25 of the straight tube portion 13c is constant for the entire length of the slope portion 31. However, the angle of the ridge 33 may change in the middle in the longitudinal direction of the slope portion 31. An example of such a configuration will be described with reference to FIG. 7A.

The protrusions 61 illustrated in FIG. 7A include slope portions 62 (each corresponding to a first slope portion) that extend for the entire length of the protrusions 61. Each slope portion 62 includes a tip-end side slope region 63 that is on the distal side of the slope portion 62 and a base-end side slope region 64 that is closer to the proximal side than the tip-end side slope region 63. The base-end side slope region 64 joins the proximal side of the tip-end side slope region 63. A slope angle of a ridge 65 to the outer surface 25 of the straight tube portion 13c in each tip-end side slope region 63 is different from a slope angle of the ridge 65 to the outer surface 25 of the straight tube portion 13c in the corresponding base-end side slope region 64 (hereinafter the slope angles of the ridges 65 to the surface 25 of the straight tube portion 13c may be referred to as slope angles of the ridges 65). Specifically, the slope angle α of the ridge 65 in the tip-end side slope region 63 is less than the slope angle β, of the ridge 65 in the base-end side slope region 64 (α<β).

Because, the slope angles of the ridges 65 in the tip-end side slope region 63 and the base-end side slope region 64 are different from each other, a variety of ways of widening the lesion site can be provided. For example, the lesion 38 may be cut by the tip-end side slope region 63 first to widen the lesion site and then by the base-end side slope region 64 to further widen the section. To provide such a variety of ways, the slope angle α of the ridge 65 in the tip-end side slope region 63 may be greater than the slope angle β of the ridge 65 in the base-end side slope region 64. As long as the slope angles of the ridges 65 are different from each other between the slope sections 63 and 64, such an advantageous effect can be achieved.

Because the slope angle of the ridge 65 in each tip-end side slope region 63 is less than the slope angle of the ridge 65 in the corresponding base-end side slope region 64, an overall height of each protrusion 61 is smaller in the tip-end side slope region 63. Therefore, the insertability of the balloon 13 is less likely to decrease. Because the slope angle of the ridge 65 in each base-end side slope region 64 is greater than the slope angle of the ridge 65 in the corresponding tip-end side slope region 63, the protrusion 61 can more easily dig into the lesion. Therefore, the lesion site further properly widens.

(3) Each protrusion 30 includes the non-slope portion 32 on the proximal side of the corresponding slope portion 31 (corresponding to a first slope portion) in the above embodiments. However, each protrusion 30 may be modified to further include a slope portion on the proximal side of the first slope portion. Examples of such a configuration will be described with reference to FIGS. 7B and 7C.

Protrusions 71 illustrated in FIG. 7B include a first slope portion 72, a second slope portion 73, and a third slope portion 74 that are arranged in line with respect to the axial direction. The first slope portion 72 includes the distal edge of the protrusion 71. The second slope portion 73 extends from the proximal edge of the first slope portion 72 toward the proximal side. The third slope portion 74 extends from the proximal edge of the second slope portion 73 toward the proximal side. The first slope portion 72 corresponds to a first slope portion and the second slope portion 73 corresponds to a second slope portion.

The ridges 75 of the protrusions 71 in the first slope portion 72, the second slope portion 73, and the third slope portion 74 are all angled to the outer surface 25 of the straight tube portion 13c. Specifically, the ridges 75 in the first slope portion 72, the second slope portion 73, and the third slope portion 74 all slope down to the outer surface 25 of the straight tube portion 13c toward the distal side. Namely, the ridges 75 are sloped in the same direction. Slope angles of the ridges 75 in the first to the third slope portions 72 to 74 are equal.

At the distal edge of the second slope portion 73, the ridge 75 is closer to the outer surface 25 of the straight tube portion 13c than the ridge 75 at the proximal edge of the first slope portion 72. Specifically, the ridge 75 at the distal edge of the second slope portion 73 is adjacent to the outer surface 25 of the straight tube portion 13c. A first corner 76 is defined by the proximal edge of the first slope portion 72 and the ridge 75 in the first slope portion 72. At the distal edge of the third slope portion 74, the ridge 75 is closer to the outer surface 25 of the straight tube portion 13c than the ridge 75 at the proximal edge of the second slope portion 73. Specifically, the ridge 75 at the distal edge of the third slope portion 74 is adjacent to the outer surface 25 of the straight tube portion 13c. A second corner 77 is defined by the proximal edge of the second slope portion 73 and the ridge 75 in the second slope portion 73. A third corner 78 is defined by the proximal edge of the third slope portion 74 and the ridge 75 in the third slope portion 74.

Because the protrusions 71 include the first to the third corners 76 to 78 at the proximal edges of the first to the third slope portions 72 to 74, respectively, the protrusions 71 can easily dig into the lesion during widening of the lesion site using the first to the third corners 76 to 78. Because each protrusion 71 includes multiple corners 76 to 78, such an advantageous effect can be easily achieved. Although each protrusion 71 illustrated in FIG. 7B includes three slope portions 72 to 74 (i.e., three corners 76 to 78), the number of the slope portions is not limited to three. Two slope portions or four or more slope portions may be included (this may apply to the example illustrated in FIG. 7C).

Protrusions 81 illustrated in FIG. 7C include first slope portions 82, second slope portions 83, and third slope portions 84. The first slope portion 82 (corresponding to a first slope portion), the second slope portion 83 (corresponding to a second slope portion), and the third slope portion 84 in each protrusion 81 are arranged in line in this sequence in the axial direction from the distal side to the proximal side. Slope angles of ridges 85 are different from one another in the first to the third slope portions 82 to 84, which is different from the example illustrated in FIG. 7B. Specifically, the slope angle of the ridge 85 in the second slope portion 83 is greater than the slope angle of the ridge 85 in the first slope portion 82. The slope angle of the ridge 85 in the third slope portion 84 is greater than the slope angle of the ridge 85 in the second slope portion 83. In the example illustrated in FIG. 7C, the closer to the proximal side the first to the third slope portions 82 to 84 are, the greater the slope angles are. A first corner 86, a second corner 87, and a third corner 88 are defined at the proximal edges of the first to the third slope portions 82 to 84, respectively. Easiness levels of digging with the first corner 86, the second corner 87, and the third corner 88 of the protrusions 81 into the lesion are different from one another. Therefore, a variety of ways of widening the lesion site can be provided.

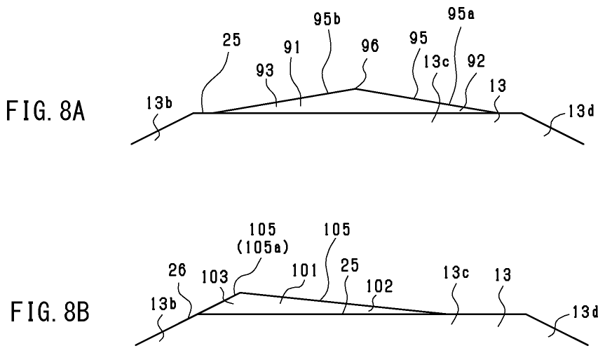

(4) Protrusions may configured to include slope portions other than the first slope portions. Each of such slope portions may be on the proximal side of the corresponding first slope portion. Ridges in the other slope portion slopes down in a different direction from the direction in which the first slope portion slopes down. Examples of such a configuration will be described with reference to FIGS. 8A and 8B.

In the example illustrated in FIG. 8A, protrusions 91 include distal-side slope portions 92 and proximal-side slope portions 93. The distal-side slope portion 92 of each protrusion 91 is on the distal side of the protrusion 91 and the proximal-side slope portion 93 of the protrusion 91 is closer to the proximal side than the distal-side slope portion 92. The lengths of the distal-side slope portion 92 and the proximal-side slope portion 93 measuring in the axial direction are equal. In the distal-side slope portion 92, the ridge 95 of the protrusion 91 slopes down to the outer surface 25 of the straight tube portion 13c toward the distal side. At the distal edge of the distal-side slope portion 92, the ridge 95 joins the outer surface 25 of the straight tube portion 13c. In the proximal-side slope portion 93, the ridge 95 of the protrusion 91 slopes down to the outer surface 25 of the straight tube portion 13c toward the proximal side. At the proximal edge of the proximal-side slope portion 93, the ridge 95 joins the outer surface 25 of the straight tube portion 13c. The distal-side slope portion 92 corresponds to a first slope portion and the proximal-side slope portion 93 corresponds to a third slope portion.

Because each protrusion 91 includes the proximal-side slope portion 93, the balloon 13 with the protrusions 91 is less likely to be caught by a vessel wall or a lesion during removal of the balloon 13 from the body. Therefore, the balloon 13 can be easily removed from the body.

In the example illustrated in FIG. 8A, the proximal-side slope portions 93 extend from the proximal edges of the respective distal-side slope portions 92 toward the proximal side. A first section of each ridge 95 (hereinafter may be referred to as the first section 95a of the ridge 95) joins a second section of the ridge 95 (hereinafter may be referred to as the second section 95b of the ridge 95) at a border between the distal-side slope portion 92 and the proximal-side slope portion 93 of each protrusion 91. A corner 96 is defined by the first section 95a of the ridge 95 and the second section 95b of the ridge 95 at the border between the distal-side slope portion 92 and the proximal-side slope portion 93 of each protrusion 91. With the corners 96, the protrusions 91 can easily dig into the lesion.

In the example illustrated in FIG. 8A, the corners 96 are located at the middle in the axial direction of the straight tube portion 13c. This is an advantageous configuration for widening the lesion site using the corners 96. The height of each protrusion 91 decreases as distances from the corner 96 increase toward the respective sides in the axial direction. According to the configuration, the protrusions 91 are less likely to contact healthy portions of the body. The corner 96 of each protrusion 91 illustrated in FIG. 8A is located at the middle of the longitudinal direction of the protrusion 91.

In the example illustrated in FIG. 8A, the distal-side slope portion 92 and the proximal-side slope portion 93 are not necessary to be joined in terms of easy removal of the balloon 13 with the proximal-side slope portion 93 from the body. Each protrusion 91 may include a non-slope portion between the distal-side slope portion 92 and the proximal-side slope portion 93. The non-slope portion may have a ridge that extends parallel to the outer surface 25 of the straight tube portion 13c.

In the example illustrated in FIG. 8B includes protrusions 101. Similar to the example illustrated in FIG. 8A, each protrusion 101 includes a distal-side slope portion 102 (corresponding to a first slope portion) on the distal side of the protrusion 101 and a proximal-side slope portion 103 (corresponding to a third slope portion) closer to the proximal side than the distal-side slope portion 102. Similar to the example illustrated in FIG. 8A, ridges 105 of the protrusions 101 in the example illustrated in FIG. 8B slope in opposite directions in the distal-side slope portions 102 and the proximal-side slope portions 103. In the example illustrated in FIG. 8B, the proximal edge of the proximal-side slope portion 103 is located at the same position as the proximal edge of the straight tube portion 13c. A slope angle of the ridge 105 in each proximal-side slope portion 103 (hereinafter may be referred to as a ridge section 105a) is equal to a slope angle of an outer surface 26 of the proximal-side tapered portion 13b (a slope angle to the axial direction). The ridge section 105b joins the outer surface 26 of the proximal-side tapered portion 13b. According to the configuration, the balloon 13 with the protrusions 101 is further less likely to be caught by the vessel wall during removal of the balloon 13 from the body.

(5) Protrusions may include cutouts in the middle. For example, each protrusion 30 in the above embodiments may include a cutout in the middle of the non-slope portion 32. The cutout may be formed such that an opening thereof is in an outer surface of the protrusion 30 and a depth measuring in a radial direction of the straight tube portion 13c. According to the configuration, the balloon 13 can be more properly advanced along the curved blood vessel during the insertion of the balloon 13 into the cured blood vessel.

(6) In the embodiments described above, the protrusions are exclusively on the straight tube portion 13c of the balloon 13. However, the protrusions may be on the proximal-side tapered portions 13b or the distal-side tapered portions 13d.

(7) In the above embodiments, the ridges 33 of the protrusions 30 join the outer surface 25 of the straight tube portion 13c at the distal edges of the slope portions 31 of the protrusions 30. However, the ridges 33 may not join the outer surface 25 of the straight tube portion 13c at the distal edges of the slope portions 31. That is, the ridges 33 may be spaced apart from the outer surface 25 of the straight tube portion 13c at the distal edges of the slope portions 31.

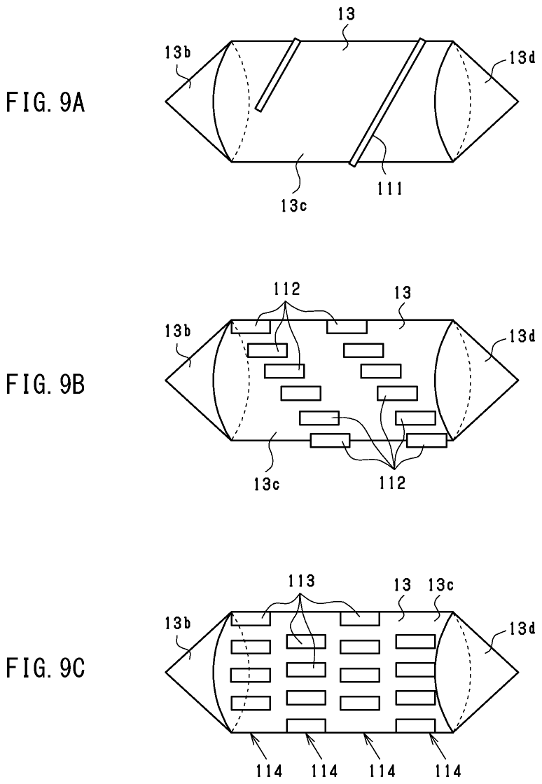

(8) The arrangement of the protrusions 30 on the straight tube portion 13c is not limited to the arrangement in the above embodiments. The protrusions 30 in the above embodiments linearly extend along the axial direction of the straight tube portion 13. However, the protrusions 30 may be modified as illustrated in FIG. 9A, that is, a protrusion 111 that spirally extends about the axis of the straight tube portion 13c. The protrusion 111 includes a slope portion (corresponding to a first slope portion) that includes a distal edge of the protrusion 111. According to the configuration, the advantageous effects described earlier can be achieved. Further, the protrusion 111 is in the entire area of the straight tube portion 13c. Therefore, the protrusion 111 can make incisions in a large area of a concentric lesion. The protrusion 111 can properly make incisions in an eccentric lesion.

As illustrated in FIGS. 9B and 9C, a large number of protrusions 112 and 113 having small lengths measuring in the axial direction may be arranged on the straight tube portion 13c. In each example, multiple protrusions 112 or 113 are arranged in the circumferential direction and the axial direction of the straight tube portion 13c. In such a configuration, the protrusions 112 or 113 are loosely arranged in the axial direction and the circumferential direction of the straight tube portion 13c. In comparison to the configuration in which the protrusions have large lengths measuring in the axial direction, the rigidity of the straight tube portion 13c is less likely to increase.

In the example illustrated in FIG. 9B, multiple protrusions 112 are spirally arranged on the straight tube portion 13c about the axis of the straight tube portion 13c. In the example illustrated in FIG. 9C, multiple lines 114 each including multiple protrusions 113 that are arranged in the circumferential direction of the straight tube portion 13c are formed. The multiple lines 114 are separated from one another in the axial direction of the straight tube portion 13c. The protrusion 113 are arranged such that every adjacent two of the protrusions 113 in adjacent two lines 114 are not aligned in the axial direction. According to the configuration, the rigidity of the straight tube portion 13c is less likely to increase.

(9) In the above embodiments, the protrusions 30 and the balloon 13 are integrally molded. However, linear parts may be prepared separately from the balloon 13 and then fixed to the outer surface 25 of the straight tube portion 13c of the balloon 13 by heat sealing or with an adhesive to extend in the axial direction. The linear parts protrude from the outer surface 25 of the straight tube portion 13c and thus the linear parts are equivalent to the protrusions.

(10) Linear parts may be prepared separately from the balloon 13 and loosely attached to the outer surface 25 of the straight tube portion 13c. Specifically, the linear parts may be made of a resin material having flexibility and stretched in the axial direction across the balloon 13 (the straight tube portion 13c) over the outer surface 25. Proximal ends of the linear parts may be bonded to the outer tube 15 and distal ends of the linear parts may be bonded to a furthermost portion of the distal end of the inner tube 16 outside the balloon 13. According to the configuration, the linear parts are disposed on the outer surface 25 of the straight tube portion 13c to extend in the axis direction when the balloon 13 is inflated. In this state, the linear parts are set to protrude from the outer surface 25 of the balloon 13. According to the configuration, the linear parts can make incisions in the lesion when the balloon 13 is inflated. In this example, portions of the linear parts that are on the outer surface 25 of the straight tube portion 13c to protrude from the outer surface 25 of the straight tube portion 13c correspond to protrusions (hereinafter referred to as the protrusions).

In the above configuration, the protrusions include first slope portions including ridges. Each ridge slopes down to the outer surface 25 of the straight tube portion 13c toward the distal end. For example, each slope portion may extend for the entire length of the protrusion. The height of the protrusion above the outer surface 25 decreases from the proximal side toward the distal side. According to the configuration, the lesion site properly widens without reducing the insertability of the balloon 13. A portion of each linear part closer to the distal side than the distal edge of the distal edge of the protrusion may have a cross section having the same shape and size as the cross section of the distal end of the protrusion.

The present disclosure has been described with reference to the embodiments. However, the present disclosure is not limited to the embodiments or the structures. Various modifications and modifications within a scope of equivalents may be within in the technical scope of the present disclosure. Further, various combinations, configurations, and other combinations and configurations including single elements, more or less, may be within the technical scope of the present disclosure.

What is claimed is:

1. A balloon catheter comprising:
a balloon that is inflatable and deflatable and disposed on a tip-end side of the balloon catheter, wherein
the balloon comprises a straight tube portion having a cylindrical shape, and
a diameter of the balloon is largest at the straight tube portion among portions of the balloon in a state where the balloon is inflated; and
a linear protrusion that protrudes from an outer surface of the straight tube portion and extends in an axial direction of the straight tube portion, wherein
the linear protrusion includes a ridge at a projected end of the linear protrusion, wherein the ridge extends in a longitudinal direction of the linear protrusion,
the linear protrusion includes a slope portion that extends over at least a part of the linear protrusion along the longitudinal direction, wherein the ridge slopes down to the outer surface in the slope portion,
the slope portion includes a first slope portion in which the ridge slopes down to the outer surface toward the tip-end side, and
the linear protrusion has a projection height from the outer surface that becomes lower toward the tip-end side in the axial direction within a region between a base-end side edge of the first slope portion and a tip-end side edge of the linear protrusion.

2. The balloon catheter according to claim 1, wherein
a tip-end side edge of the first slope portion matches the tip-end side edge of the linear protrusion, and
the ridge joins the outer surface at the tip-end side edge of the first slope portion.

3. The balloon catheter according to claim 1, wherein
the linear protrusion includes a non-slope portion that joins the base-end side edge of the first slope portion, and
the ridge in the non-slope portion extends parallel to the outer surface.

4. The balloon catheter according to claim 2, wherein
the linear protrusion includes a non-slope portion that joins the base-end side edge of the first slope portion, and
the ridge in the non-slope portion extends parallel to the outer surface.

5. The balloon catheter according to claim 1, wherein
the tip-end side edge of the linear protrusion is closer to a base-end side than is the tip-end side edge of the straight tube portion,
the straight tube portion includes a no-protrusion region that does not include the linear protrusion, and
the no-protrusion region is closer to the tip-end side than is the linear protrusion.

6. The balloon catheter according to claim 2, wherein
the tip-end side edge of the linear protrusion is closer to a base-end side than is the tip-end side edge of the straight tube portion,
the straight tube portion includes a no-protrusion region that does not include the linear protrusion, and
the no-protrusion region is closer to the tip-end side than is the linear protrusion.

7. The balloon catheter according to claim 3, wherein
the tip-end side edge of the linear protrusion is closer to a base-end side than is the tip-end side edge of the straight tube portion,
the straight tube portion includes a no-protrusion region that does not include the linear protrusion, and
the no-protrusion region is closer to the tip-end side than is the linear protrusion.

8. The balloon catheter according to claim 2, wherein
the linear protrusion includes a non-slope portion that joins the base-end side edge of the first slope portion,
the ridge in the non-slope portion extends parallel to the outer surface in the non-slope portion,
the tip-end side edge of the linear protrusion is closer to a base-end side than is the tip-end side edge of the straight tube portion,
the straight tube portion includes a no-protrusion region that does not include the linear protrusion, and
the no-protrusion region is closer to the tip-end side than is the linear protrusion.

9. The balloon catheter according to claim 1, wherein
the first slope portion includes a tip-end side slope region and a base-end side slope region that is disposed closer to a base-end side than is the tip-end side slope region, and
a slope angle of the ridge to the outer surface in the tip-end side slope region is different from a slope angle of the ridge to the outer surface in the base-end side slope region.

10. The balloon catheter according to claim 2, wherein
the first slope portion includes a tip-end side slope region and a base-end side slope region that is disposed closer to a base-end side than is the tip-end side slope region, and
a slope angle of the ridge to the outer surface in the tip-end side slope region is different from a slope angle of the ridge to the outer surface in the base-end side slope region.

11. The balloon catheter according to claim 9, wherein the slope angle of the ridge in the tip-end side slope region is smaller than the slope angle of the ridge in the base-end side slope region.

12. The balloon catheter according to claim 10, wherein the slope angle of the ridge in the tip-end side slope region is smaller than the slope angle of the ridge in the base-end side slope region.

13. The balloon catheter according to claim 1, wherein
the slope portion further includes a second slope portion that extends from the base-end side edge of the first slope portion toward a base-end side,
the ridge in the second slope portion slopes down in a direction same as a direction in which the ridge in the first slope portion slopes down, and
the ridge at a tip-end side edge of the second slope portion is closer to the outer surface than is the ridge at the base-end side edge of the first slope portion.

14. The balloon catheter according to claim 2, wherein
the slope portion further includes a second slope portion that extends from the base-end side edge of the first slope portion toward a base-end side,
the ridge in the second slope portion slopes down in a direction same as a direction in which the ridge in the first slope portion slopes down, and
the ridge at a tip-end side edge of the second slope portion is closer to the outer surface than is the ridge at the base-end side edge of the first slope portion.

15. The balloon catheter according to claim 1, wherein
the slope portion further includes a third slope portion that is closer to a base-end side than is the first slope portion, and
the ridge in the third slope portion slopes down to the outer surface toward the base-end side.

16. The balloon catheter according to claim 2, wherein
the slope portion further includes a third slope portion that is closer to a base-end side than is the first slope portion, and
the ridge in the third slope portion slopes down to the outer surface toward the base-end side.

17. The balloon catheter according to claim 15, wherein
the third slope portion extends from the base-end side edge of the first slope portion toward the base-end side, and
the ridge in the third slope portion joins the ridge in the first slope portion at a border between the first slope portion and the third slope portion.

18. The balloon catheter according to claim 2, wherein
the slope portion further includes a third slope portion that is closer to a base-end side than is the first slope portion,
the ridge in the third slope portion slopes down to the outer surface toward the base-end side,
the third slope portion extends from the base-end side edge of the first slope portion toward the base-end side, and
the ridge in the third slope portion joins the ridge in the first slope portion at a border between the first slope portion and the third slope portion.
